(19) United States
(12) Patent Application Publication
(10) Pub. No.: US 2019/0076050 A1
(43) Pub. Date: Mar. 14, 2019

(54) ADJUSTMENT OF A COUCH POSITION OF A TOMOGRAPH

(71) Applicant: Siemens Healthcare GmbH, Erlangen (DE)

(72) Inventors: Miriam KEIL, Erlangen (DE); Manuela RICK, Uttenreuth (DE)

(73) Assignee: Siemens Healthcare GmbH, Erlangen (DE)

(21) Appl. No.: 16/127,437

(22) Filed: Sep. 11, 2018

(30) Foreign Application Priority Data
Sep. 13, 2017 (EP) 17190821.3

(51) Int. Cl. A61B 5/055; A61B 5/00; G01R 33/54; G06F 3/0484

(52) U.S. Cl. CPC A61B 5/0555; G06F 3/04842; G01R 33/543; A61B 5/7435

(57) ABSTRACT

A method is disclosed for adjusting a couch position, the method including: acquiring a user selection of a scanning protocol from a plurality of possible scanning protocols; acquiring a user input in respect of an isocenter position; acquiring a user input in respect of a focus position; displaying an overview of the body region to be examined, including a selection range and an indicator of the focus position relative to the body region, the selection range being displayable aligned with the focus position; acquiring a user input in respect of a changed focus position; aligning the selection range with the changed focus position; and adjusting the couch position to a focus couch position corresponding to the focus position upon the changed focus position meeting a test criterion, and adjusting to an isocenter couch position corresponding to the isocenter position upon the changed focus position not meeting the test criterion.

### PRIORITY STATEMENT

The present application hereby claims priority under 35 U.S.C. § 119 to European patent application number EP17190821.3 filed Sep. 13, 2017, the entire contents of which are hereby incorporated herein by reference.

### FIELD

At least one embodiment of the invention relates to a method for adjusting a couch position for a couch of a tomograph for scanning a body region of an examination object situated on the couch. A corresponding magnetic resonance tomograph, a corresponding arithmetic unit, a computer program and a computer-readable data carrier also form part of embodiments of the invention.

### BACKGROUND

The examination object or also patient is usually a human or a human body. In principle, the patient can also be an animal. The two terms “examination object” and “patient” will therefore be used synonymously below. The examination object can alternatively be a plant or a non-living object made of metal or rock, for example a historical artifact or the like.

A scan or examination is taken to mean the obtaining of image data of a body region of the examination object via the tomograph. An examination begins with a body being laid on said couch and the couch then being moved into a couch position or a couch position being adjusted at which the body region to be examined has an advantageous position in respect of an isocenter of the tomograph.

In particular the spatial region having the highest quality imaging performance or imaging fidelity of the tomograph is designated the isocenter. The isocenter can therefore be for example the center of the calibrated spatial region of the tomograph. By default, within the sense of optimum image quality, a couch position is consequently adjusted in which the body region to be examined is arranged as close as possible to the isocenter, ideally exactly in the isocenter. This couch position will, hereinafter and within the context of embodiments of the invention, be designated the isocenter couch position.

Following positioning of the examination object in the tomograph the actual examination takes place, that is the generation of scans or image data in a manner known per se.

Once the desired image data of the body region and sometimes further body regions has been obtained, the body can be removed from the couch again, in other words for example the patient can leave the couch. The examination is ended thereby.

The examination can be conducted or monitored by a user, for example a doctor or assistant (MTA—medical technical assistant). The user can firstly select an examination program for the examination object arranged on the couch; the selection can depend on a medical issue or a known clinical picture. An examination program can be divided into examination steps. When an examination program is activated, a control unit of the tomograph can for example prompt the user to select or mark a body part or body region or a position on the body of the patient, for example the liver or the heart, for a first examination step by means of laser markers of the tomograph. By way of this interaction the user can determine at which position on the patient's body the user wants image data to be generated, and this is then ideally arranged in the isocenter of the tomograph during the scan. The isocenter position, in other words the position on the body of the patient marked by the user and which is to be imaged, will be referred to hereinafter in this regard.

The laser marker is arranged for example on the tomograph housing above the coil opening and emits a laser beam vertically downwards. For the marking process the couch with a patient situated thereon is moved in such a way that the body position of interest comes to rest in the laser beam. Since the position of laser marker or laser beam relative to isocenter of the tomograph is previously defined or known, the isocenter couch position may now also be derived and adjusted. It can be provided that with an adjusted isocenter couch position an overview, what is known as a localizer image, is generated which can be used during subsequent protocol planning and optionally to check the isocenter couch position defined by the user.

In a second examination step it can be provided that the user marks or selects a focus position, in other words a second body position in the localizer image of the patient, in particular one that differs from the isocenter position. The focus position can therefore be located for example over a particular organ or a specific anatomical structure which the user wishes to image centrally with the scan. This is advantageous if the selection of the isocenter position in the overview proves to inaccurate or incorrect or one of the following scanning steps does not require maximum image quality. A scan can then also occur outside of the isocenter position, for example the focus position. This advantageously reduces or minimizes couch movements and provides for greater comfort during the scan. Since the relative position of focus position and isocenter position is known, a corresponding focus couch position can also be inferred in which the couch is adjusted such that the patient situated thereon comes to rest with the focus position in the isocenter of the tomograph. The selection of the couch position to be adjusted during a scan is supplied to the user within the context of what is known as the Scan@Center concept.

In a further examination step the user can select a scanning protocol by means of which image data is to be generated. A scanning protocol is a set of all parameters which have to be adjusted at the tomograph in order to generate the required image data for the body region. The user then activates the scan, in other words the selected scanning protocol is carried out. This produces the image data from the selected or marked body region. Obtaining image data of a different body region or image data of the same body region comprising different image information, for example a different contrast, etc., can thereafter be provided in a next examination step. Once all examination steps have been completed, in each case using a scanning protocol provided for this purpose, the examination program is finished. The examination is therefore finished. The order of examination steps constitutes a work sequence or workflow for the user.

As all parameters of the tomograph required for obtaining image data of a body region are combined to form a scanning protocol, it can be ensured that the best or a requisite image quality is attained without the user having to worry about technical details.

When planning each individual examination step, also called protocol or slice planning, the user can finely adjust the isocenter or focus position, for example using the localizer image mentioned above or a comparable scan, in other words the position in the body region which is to be arranged in the isocenter for the scan.

Until now, the user has for this purpose been shown in the localizer image a selection range to be imaged corresponding to a field of view of the tomograph or corresponding to a planned slice group that is always aligned with the isocenter position. In other words, a center of the planned slice group is shown overlaid or centered on the isocenter position. By default this occurs independently of whether the user initially input a desired focus position or not. In other words, information about a focus position is not taken into account during slice planning. The result of this is that the planning of every individual examination step optionally requires not just complex manual adjustment of the selection range or the slice group center to the focus position, but also more and possibly longer adjusting steps are necessary in order to achieve the couch position selected for the scan. Preparation times are extended and patient comfort is reduced as a result. In addition, the examination times are lengthened, limiting the number of patients examined per day.

### SUMMARY

Embodiments of the invention provide alternative device(s)/method(s) which allow adjustment of a couch position to be simplified, to be expedited and a higher level of comfort to be attained for a patient during a scan.

Preferred and/or alternative, advantageous variants and embodiments are the subject matter of the claims.

Embodiments of the invention include methods and devices. Features, advantages or alternative embodiments mentioned in this connection can be transferred to the other subject matter embodiments and vice versa. In other words, the embodiments in question (which are directed for example at a method) are also developed by the features which are described or claimed in connection with one of the devices. The corresponding functional features of the method are formed by corresponding concrete modules or units.

In an embodiment, the invention relates to a method for adjusting a couch position for a couch of a tomograph for scanning a body region of an examination object situated on the couch, the method comprising:

acquiring a user selection of a scanning protocol from a plurality of possible scanning protocols;
acquiring a user input in respect of an isocenter position;
acquiring a user input in respect of a focus position;
displaying an overview of the body region to be examined, including a selection range and an indicator of the focus position relative to the body region, the selection range being displayable aligned with the focus position;
acquiring a user input in respect of a changed focus position;
aligning the selection range with the changed focus position; and
adjusting the couch position to a focus couch position corresponding to the focus position upon the changed focus position meeting a test criterion, and adjusting to an isocenter couch position corresponding to the isocenter position upon the changed focus position not meeting the test criterion.

In an embodiment, the invention relates to an arithmetic unit for adjusting a couch position for a couch of a tomograph for examining a body region of an examination object situated on the couch, the arithmetic unit comprising:

a memory storing program computer-readable instructions; and
one or more processors configured to execute the instructions such that the one or more processors are configured to,
acquiring a user selection of a scanning protocol from a plurality of possible scanning protocols,
acquiring a user input in respect of an isocenter position,
acquiring a user input in respect of a focus position,
displaying an overview of the body region to be examined, including a selection range and an indicator of the focus position relative to the body region, the selection range being displayable aligned with the focus position,
acquiring a user input in respect of a changed focus position,
aligning the selection range with the changed focus position, and
adjusting the couch position to a focus couch position corresponding to the focus position upon the changed focus position meeting a test criterion, and adjusting to an isocenter couch position corresponding to the isocenter position upon the changed focus position not meeting the test criterion.

In a further embodiment, the invention relates to an arithmetic unit for adjusting a couch position for a couch of a tomograph for examination of a body region of an examination object situated on the couch, including at least one processor for carrying out an embodiment of the inventive method.

In a further embodiment, the invention relates to a non-transitory computer program data carrier storing program code in order to carry out an embodiment of the inventive method for adjusting a couch position for a couch of a tomograph for examination of a body region of an examination object situated on the couch when the program code is run on a computer.

In a further embodiment, the invention relates to non-transitory a computer-readable medium having program code of a computer program in order to carry out an embodiment of the inventive method for adjusting a couch position for a couch of a tomograph for examination of a body region of an examination object situated on the couch when the program code is run on a computer.

In a further embodiment, the invention also relates to a tomograph for adjusting a couch position for a couch of a tomograph for examination of a body region of an examination object situated on the couch, having an inventive arithmetic unit. The tomograph is preferably a magnetic resonance tomograph. The arithmetic unit is advantageously integrated in the medical imaging system. Alternatively, the arithmetic unit can also be arranged at a distance or remotely. The arithmetic unit can be designed to carry out the inventive method for a tomograph or for a plurality of medical systems, for example in a radiology center or hospital comprising a plurality of magnetic resonance tomographs.

### BRIEF DESCRIPTION OF THE DRAWINGS

The above-described properties, features and advantages of this invention and the manner in which they are achieved will become clearer and more comprehensible in connection with the following description of the example embodiments which will be illustrated in more detail in connection with the drawings. This description does not limit the invention to these example embodiments. In various figures identical components are provided with identical reference characters. As a rule, the figures are not to scale. In the drawings:

FIG. 1 shows a view of an inventive tomograph in the form of a magnetic resonance tomograph according to an example embodiment,

FIG. 2 shows a schematic diagram of an inventive method according to an example embodiment,

FIG. 3 shows an overview of a body region to be examined according to an example embodiment of the invention,

FIG. 4 shows a further overview of a body region to be examined according to a further example embodiment of the invention, and

FIG. 5 shows a further overview of a body region to be examined according to a further example embodiment of the invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

In a first embodiment, the present invention relates to a method for adjusting a couch position for a couch of a tomograph for scanning a body region of an examination object situated on the couch. The method comprises a number of steps.

A first step comprises acquisition known per se of a user selection of a scanning protocol from a plurality of possible scanning protocols. The plurality of scanning protocols together forms a scanning program. Acquisition can comprise receiving or obtaining a user input or user selection which can take place by way of an output and input unit of the tomograph.

A second step comprises acquisition or receiving known per se of a user input in respect of an isocenter position and a third step comprises acquisition or receiving known per se of a user input in respect of a focus position, as already described in the introduction.

It should be mentioned once again that the isocenter position can be defined for example by means of laser markers. In this sense acquisition also comprises manual positioning of the patient under the laser beam by the user. The focus position can be marked in a localizer image for example at the start of a scan. The focus position identifies for example an organ or a particular anatomical structure. The focus position defines where the slice center of a scanning step should be arranged relative to the anatomy of the patient and therefore also defines the focus couch position into which the couch has to be moved for the scan. As a rule, the focus position does not differ from the isocenter position.

In a fourth step, an overview of the body region to be examined is displayed, comprising a selection range and an indicator of the focus position relative to the body region, wherein the selection range is displayed aligned with the focus position. The overview can be for example the localizer image or a comparable image of the body region to be examined. The display preferably occurs for the user for example by way of an output unit of the tomograph. The display can however also be transferred to a remote output unit, for example a monitor, and be displayed there, the output unit being arranged for example in a side room. The output unit can also be designed for example as a portable output unit, for example a tablet, or so the user can move during protocol planning.

The selection range represents some of the body region to be examined which is imaged by the examination predefined by the scanning protocol. The selection range can correspond to a field of view of the tomograph. According to the invention, display of the selection range can also include display of the planned slice group, in other words a display of the position, width, number, distribution, etc. of the slices examined by the scan. In particular, according to the invention, a display of the slice group center can be included.

The indicator of the focus position corresponds to a visual marking of the focus position initially defined by the user relative to the imaged anatomy in the body region to be examined, for example in the form of any formed line, but also in the form of a triangle arranged at the edge of the overview, with a peak of the triangle marking the focus position.

The display can also comprise a further indicator for illustrating the position of the isocenter position, for example likewise in the form of a line.

The inventors have therefore recognized that, contrary to current practice, it can be advantageous to use the focus position instead of the isocenter position as the initial display position for the selection range during protocol planning. In other words, when displaying the overview, the selection range is inventively arranged such that the center of the selection range or a corresponding slice group center and the focus position are overlaid. In other words, the selection range is displayed centered on the focus position.

In step five of an embodiment of the inventive method, a user input is acquired in respect of a changed focus position. The changed focus position can match the initially selected focus position but can also differ therefrom. The user input can consequently be made in the form of a confirmation of the focus position or in the form of a correction of the focus position.

In a sixth method step the selection range is aligned with the changed focus position. Steps five and six preferably take place together by, for example, the user shifting the selection range or the center of the selection range or the slice center to the desired, changed focus position or confirming the focus position in the overview by way of the output and input unit of the tomograph. In the latter case alignment of the selection range comprises retaining its position. Alternatively the user can select a changed focus position by inputting coordinates in respect of a reference coordinate system of the tomograph by means of the input unit and shifting the selection range automatically in the overview to this location.

In step seven of an embodiment, the couch position is adjusted to a focus couch position corresponding to the focus position if the changed focus position meets a test criterion, and is adjusted to an isocenter couch position corresponding to the isocenter position if the changed focus position does not meet the test criterion. This step therefore comprises testing the changed focus position with regard to a test criterion.

The test criterion is previously defined and, generally speaking, cannot be changed by the user. If testing of the changed focus position produces a positive result, in other words the criterion is met, the couch is moved or brought into the focus couch position or remains there if the couch is already in the focus couch position for a prior examination step. The couch is only brought into the isocenter couch position for cases in which the test delivers a negative result, in other words the test criterion is not met. The test criterion itself is system-specific and can only be edited by the user with appropriate qualifications, authorization and/or experience.

The inventors have therefore recognized that, contrary to current practice, it can be advantageous to use the focus position instead of the isocenter position as the initial display position for the selection range during protocol planning. In other words, when displaying the overview, the selection range is inventively arranged such that the center of the selection range or a corresponding slice group center and the focus position are overlaid. In other words, the selection range is displayed centered on the focus position.

In a large number of applications the desired focus position will match the initial focus position or differ only minimally therefrom. According to an embodiment of the invention, the planning effort is therefore reduced since now no, or only minimal adjustments of the selection range have to be made for the scan. Protocol planning is expedited and couch movements before a scan are reduced or even superfluous.

In one example embodiment of the invention, the test criterion takes into account a distance measurement between the changed focus position and the isocenter position and is met if the distance measurement does not exceed a previously defined upper limit. In other words, the test criterion takes into account a position between body region to be examined, in particular the changed focus position defined therein, relative to the isocenter of the tomograph if a couch position corresponding to the changed focus position, in other words a second focus couch position, were to be adjusted. In other words, the test criterion takes into account a relative position between a planned slice center and isocenter of the tomograph. Only if this distance adheres to an upper limit is the focus couch position adjusted as the couch position for the scan.

In a further example embodiment of the invention, the test criterion takes into account a distance measurement between a point of the selection range furthest away from the isocenter position and the isocenter position and is met if the distance measurement does not exceed a previously defined upper limit. Information about the extent of the slice group flows into this distance measurement. In other words, consideration is given to how large the distance is between the isocenter and a body part of the body region to be examined that has just been imaged by a scan if a couch position corresponding to the changed focus position, in other words a second focus couch position, were to be adjusted. If this distance adheres to an upper limit, the focus couch position is adjusted as the couch position for the scan.

In a further example embodiment of the invention, the previously defined upper limit is dependent on an item of anatomical information and/or an item of information in respect of the homogeneity of a magnetic field of the tomograph. This can firstly take into account that some body regions can still be imaged with a predetermined minimum image quality even at a greater distance from the isocenter than can be the case with other body regions. Secondly, this takes into account at what distance, starting from the isocenter, images can still be produced with a predefined minimum quality. This is determined or influenced by the homogeneity of the magnetic field. The upper limit is therefore advantageously adapted to the anatomy and/or the homogeneity of the magnetic field or imaging performance of the tomograph.

In a further example embodiment, the overview of the body region to be examined is displayed with a selection range aligned with the focus position if an operating mode adjusted for the selected scanning protocol provides for this. In other words, according to an embodiment of the invention, an operating mode is taken into account. If, by contrast, a scanning protocol was selected whose operating mode prohibits inventive displaying of the selection range, a display can for example occur in which the selection range is aligned with the isocenter position.

An operating mode defines whether a scanning protocol or a scanning step has to be carried out while maintaining the isocenter couch position or whether couch positions that differ therefrom, for example the focus couch position or other couch positions, can be adopted. Any scanning program comprising a large number of scanning protocol also comprises an indication in the form of a global position parameter applicable to all included scanning protocols. The global position parameter therefore defines an operating mode for all scanning protocols. The global positioning parameter is adjusted during the definition of an examination program and is essentially dependent on a medical issue, the body region being considered and/or particular anatomical conditions of an examination object.

One possible operating mode is for example the LOC mode. It provides that the focus couch position is adjusted if the focus position maintains a defined maximum distance from the isocenter position for a scanning step being considered. Only if the maximum distance is exceeded is the isocenter couch position adjusted for the scan in order to avoid undesirable losses in quality in the generated scans. A further operating mode is the ISO mode. Here the isocenter couch position has to be adopted for each scanning step.

Accordingly, the overview would be inventively displayed if the LOC mode were adjusted as the operating mode. In the ISO mode a display with selection range aligned with the isocenter position would occur.

For example the parameter map of the scanning program or the global position parameter can be read out to determine the operating mode.

A further position parameter is included on the parameter map and this indicates aligned with which position a selection region or a slice group should be displayed for planning. The option ‘focus position’ is inventively provided and pre-set for this position parameter, so, if the operating mode allows this, the selection range is initially open at the focus position during planning of a scanning step.

According to a further example embodiment of the invention, it can be provided that displaying the overview of the body region to be examined occurs with a selection range aligned with a position that differs from the focus position according to a user input if the adjusted operating mode allows this. In other words, in this embodiment the possibility exists for the user to adapt the position, at which the selection range is displayed in the overview, right at the start of slice planning and/or during it. As a prerequisite for this, here too an operating mode has to be adjusted for the scanning protocol which allows this adjustment, for example the LOC mode.

According to a further example embodiment of the invention, a position that differs from the focus position can be any previously defined position within the displayed body region to be examined, in particular also the isocenter position. The user input can occur for example by output and input unit of the tomograph by way of a drop-down menu having a plurality of previously defined alternative display positions or by way of the input of coordinates in respect of a reference coordinate system of the tomograph.

According to a further example embodiment of the invention, acquisition of a user input in respect of an adaptation of the operating mode adjusted for the selected scanning protocol, and an adaptation of the operating mode corresponding to the user input is also included. In other words, according to the invention the operating mode of a scanning protocol or a scanning program can be changed to bring about the inventive display of the overview. The operating mode is preferably changed globally for all scanning protocols of a scanning program. For example, the user can inventively change an adjusted ISO mode to an LOC mode.

In a preferred example embodiment, displaying the overview of the body region to be examined comprises an indicator which displays the couch position to be adjusted as a function of the test criterion. In other words, the overview simultaneously comprises a display of whether the focus couch position or the isocenter couch position would be adjusted for the planned scan as a function of the currently adjusted changed focus position. In this way the test result is illustrated directly and intuitively for the user. The indicator can be designed for example in the form of an additional button which is filled with different colors depending on which couch position is adjusted. Alternatively, the indicator can be formed by the triangle identifying the focus position, which is either empty or filled as a function of the couch position to be adjusted. Alternative embodiments of the indicator are similarly possible.

In a further embodiment, the invention relates to an arithmetic unit for adjusting a couch position for a couch of a tomograph for examination of a body region of an examination object situated on the couch, having at least one processor for carrying out an embodiment of the inventive method.

In a further embodiment, the invention relates to a computer program having program code in order to carry out an embodiment of the inventive method for adjusting a couch position for a couch of a tomograph for examination of a body region of an examination object situated on the couch when the computer program is run on a computer.

In a further embodiment, the invention relates to non-transitory a computer-readable data carrier having program code of a computer program in order to carry out an embodiment of the inventive method for adjusting a couch position for a couch of a tomograph for examination of a body region of an examination object situated on the couch when the computer program is run on a computer.

In a further embodiment, the invention also relates to a tomograph for adjusting a couch position for a couch of a tomograph for examination of a body region of an examination object situated on the couch, having an inventive arithmetic unit. The tomograph is preferably a magnetic resonance tomograph. The arithmetic unit is advantageously integrated in the medical imaging system. Alternatively, the arithmetic unit can also be arranged at a distance or remotely. The arithmetic unit can be designed to carry out the inventive method for a tomograph or for a plurality of medical systems, for example in a radiology center or hospital comprising a plurality of magnetic resonance tomographs.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

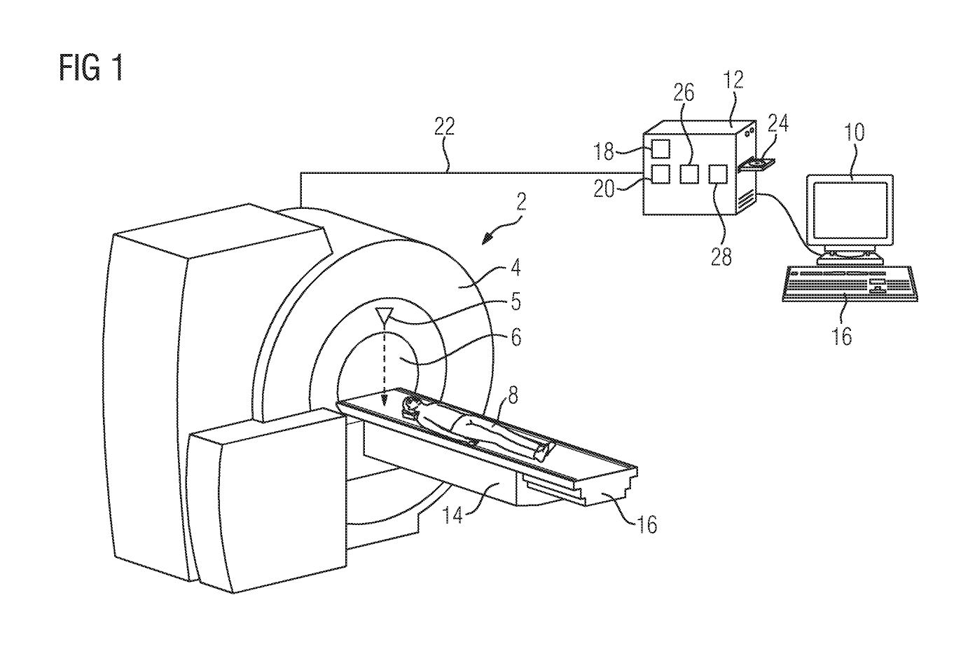

The inventive tomograph 2, of an embodiment shown in FIG. 1 in the form of a magnetic resonance tomograph comprises a hollow cylindrical base unit 4 in whose interior, what is known as the tunnel 6, an electromagnetic field is generated during operation for a magnetic resonance scan or examination of an examination object in the form of a patient 8. Furthermore, an examination couch 14 having a movable board 16 is provided. The patient 8 can be positioned on the board 16 for example as illustrated. The examination couch 14 is positioned outside of the base unit 4 such that the board 16 together with patient 8 can be moved at least partially into the tunnel 6 for the examination. Arranged on the housing of the base unit 4 above the entrance to the tunnel 6 is a laser marker 5 or a marking laser in the form of a laser source whose laser beam (broken-line arrow) propagates vertically downward onto the examination table 16. The laser marker 6 can be used for inventively defining the isocenter position.

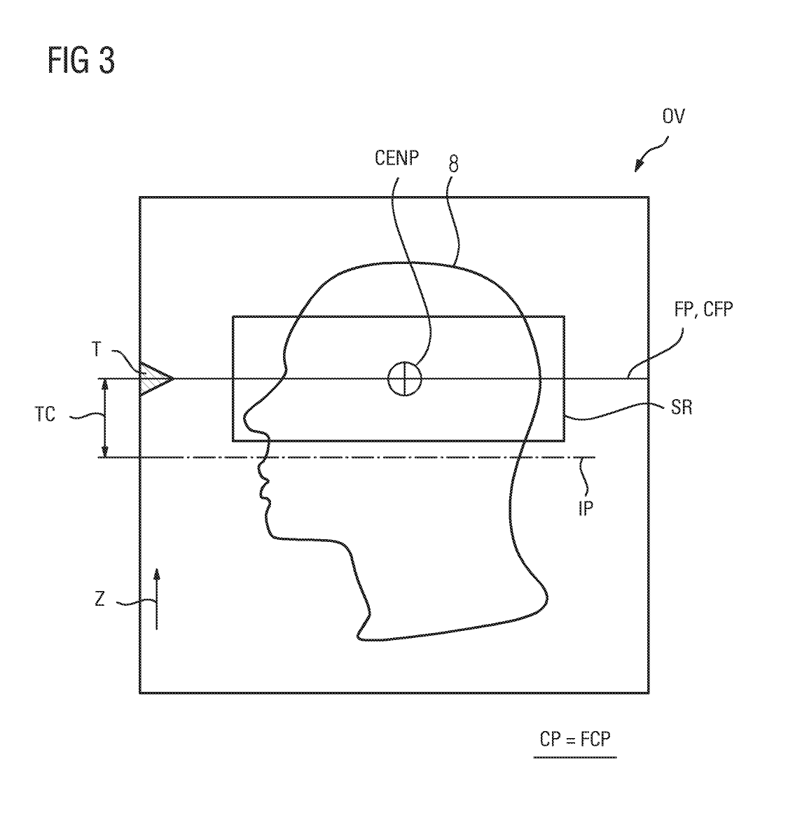

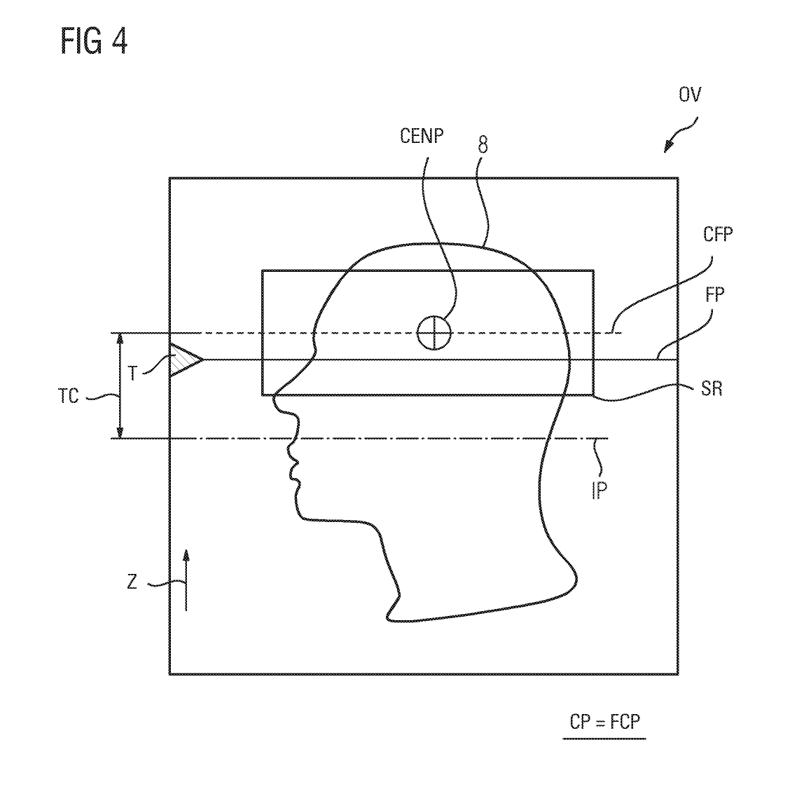

The tomograph 2 has at least one embodiment of an inventive arithmetic unit in the form of a computer system 12 which is designed as a computer and for carrying out at least one embodiment of the inventive method. The arithmetic unit 12 is accordingly connected to a display unit 10, for example for graphical display of an inventive overview, such as, for example in FIG. 3 or FIG. 4, and to an input unit 16. The display unit 10 can be for example an LCD, plasma or OLED screen. It can also be a touch-sensitive screen which is also designed as an input unit 16.

A touch-sensitive screen of this kind can be integrated in the imaging device or be designed as part of a mobile and/or portable device. The input unit 10 is for example a keyboard, a mouse, what is known as a touch screen or even a microphone for speech input. The input unit 16 can also be adapted to detect movements of a user and translate them into corresponding commands. Various positions, which can be predefined by the user, in particular the isocenter position, the focus position and/or a changed focus position, can be inventively detected and transmitted to the arithmetic unit 12 wirelessly or wired in a known manner by way of the display unit 10 and/or the input unit 16. In addition, user-side indications about the change in an operating mode can also be detected by way of the display unit 10 and/or the input unit 16 and be supplied to the arithmetic unit 12.

The computer system is also connected to the base unit 4 of the tomograph 2 for data exchange. For example, control signals for the tomograph 2 for the magnetic resonance examination can be transferred from the computer system 12 to the base unit 4. For this purpose, for example different scanning protocols, in each case coordinated with a type of examination, can be stored in a storage device 18 and be selected by a user, for example a doctor or medically-technically trained staff, before the scan according to a medical issue. For this purpose, various scanning protocols, in particular various scanning protocols of a scanning program, can be output to the user for selection via the display unit 10 and the user can select one of the scanning protocols via the input unit 16, for example by a mouse click. Alternatively, a scanning protocol can also be supplied at the tomograph 2 automatically or remotely by a remote user via an Intranet or Internet by a central default unit (not shown) by way of appropriate data links. The tomograph 2 is adjusted for the examination using the selected scanning protocol. In particular, the inventively determined couch position is adjusted at the tomograph 2. The base unit 4 is controlled according to the selected scanning protocol. On the other hand, recorded scan data is acquired for further processing in a reconstruction unit 20 (not described). The connection 22 is implemented wired or wirelessly in a known manner by way of appropriate interfaces.

The computer system 12 also comprises a determining unit 26. This is adapted to transfer the user-side inputs in respect of an isocenter position and/or a focus position, optionally by taking into account a relative position of the laser marker 5 and an isocenter of the tomograph 2 in relation to a reference coordinate system of the tomograph for a patient 8 situated on the examination table 16, into an isocenter couch position and a focus couch position respectively.

The computer system 12 also comprises a test unit 28 which is used firstly to determine an operating mode for a selected scanning protocol and, as a function of the result, to define aligned with which position a selection range should be displayed in an overview. Secondly, the test unit 28 is used to test whether an upper limit for a test criterion in respect of a changed focus position is adhered to or not met. In particular, the test criterion can be a distance measurement between isocenter position and changed focus position. In other words, a distance between the isocenter and a body position of the patient corresponding to the changed focus position is considered if a couch position corresponding to the changed focus position were to be adjusted.

Alternatively, a distance between the isocenter position and a position as far away as possible from the isocenter position within the selection range is considered. The upper limit can for example be stored for the test unit 28 so as to be retrievable in the storage device 18 of the tomograph 2. The upper limit is in particular dependent on the selected scanning protocol, in other words the underlying medical issue, the anatomical region associated therewith and/or the technical conditions of the tomograph 2, in particular the extent of the homogeneous part of its magnetic field. The test unit 28 is also set up to generate control signals for the tomograph 2 as a function of the test result and to transmit them thereto, by means of which the couch position is adjusted for the planned scan. For example, as a control signal the test unit 28 can output that the focus couch position should be adjusted if the test criterion was met, in other words the distance upper limit was maintained. If the distance upper limit is exceeded or not met, as a control signal the test unit 28 outputs that the isocenter couch position should be adjusted.

In the present case, in particular said units 26 and 28 are designed as separate modules within the computer system 12, which, where necessary, can exchange data with each other. Alternatively, all of said units can be integrated as an arithmetic unit, be it in the form of physical or functional integrity.

The computer system 12 can interact with a computer-readable data carrier 24, in particular in order to carry out an embodiment of an inventive method by a computer program having program code. Furthermore, the computer program can be retrievably stored on the machine-readable carrier. In particular, the machine-readable carrier 24 can be a CD, DVD, Blu-Ray disk, a memory stick or a hard disk.

In particular, the units 26 and 28 can be designed in the form of hardware or in the form of software. For example, the units are designed as what are known as FPGAs (acronym for “Field Programmable Gate Array”) or comprise an arithmetic logic unit.

At least one computer program can be stored on the storage device 18 of the computer system 12, and this carries out all method steps of an embodiment of the inventive method when the computer program is run on the computer. The computer program for carrying out the method steps of an embodiment of the inventive method comprises program code. Furthermore, the computer program can be configured as an executable file and/or be stored on a different computing system from the computer system 12. For example, the magnetic resonance tomograph 2 can be designed such that the computer system 12 loads the computer program for carrying out an embodiment of the inventive method via an Intranet or the Internet into its internal working memory. Alternatively it can be provided that the computer system 12 itself is part of an Internet or Intranet, for example an HIS (Hospital Information System) or an RIS (Radiology Information System) and has access to input, selected or centrally stored scanning protocols of various magnetic resonance tomographs 2 of the facility in order to carry out the inventive method centrally for various tomographs 2.

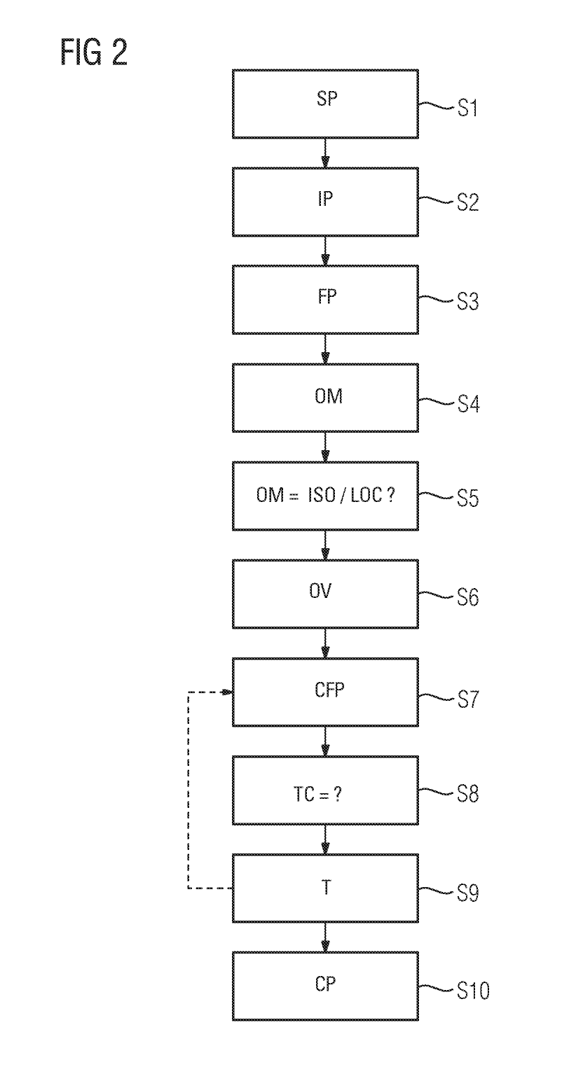

An embodiment of the inventive method shown in FIG. 2 comprises a plurality of steps. It is used for simplified and expedited adjustment of a couch position CP of a tomograph 2 for an examination. The core of the inventive method is to align selection range SR or field of view of the tomograph 2 displayed during protocol planning and which, generally speaking, also comprises the slice group that is to be acquired with a scanning protocol SP, with a previously defined focus position FP. In this way the user does not need to make any adaptations, or only minor ones, during planning to the couch position to be adjusted. This expedites scan planning and increases the comfort level for the patient 8 who is situated on the examination table 16 of the couch 14 during planning of each scanning step.

Steps S1, S2 and S3 of the method are known per se and will therefore only be briefly illustrated. In step S1 a scanning protocol SP is selected from a plurality of scanning protocols. As a rule, the plurality of scanning protocols SP corresponds to all scanning protocols encompassed by a scanning program for examination of the patient 8 in order to address a specific medical issue. The selection can be made by a user, for example by selection from a list or by automatic suggestion and user confirmation of the like. In step S2 an isocenter position IP is defined or determined for the selected scanning protocol SP. This can occur for example by means of the above-described laser marker 5 of the tomograph 2. The user therefore marks the part, region, section or an organ which is to be arranged in the isocenter of the tomograph 2 during the scan.

In step S2 an overview OV can also be acquired with the tomograph 2. The overview OV images the body region to be examined in that the couch position CP is adjusted to an isocenter couch position ICP corresponding to the isocenter position IP, which the user has marked in advance. This is also called the O-position. The isocenter couch position ICP is also called the O-position of the patient 8. The overview OV can therefore be used to check the isocenter position IP.

In step S3 a focus position FP that is different from the isocenter position IP is defined by the user. By definition, the focus position FP is the position relative to the anatomy of the patient 8 in a body region to be examined on which an examination is to be carried out. For this purpose, the user can be shown the overview OV, together with isocenter position IP, for example via an interaction interface, for example formed by the display unit 10 and the input unit 16, for example a Graphical User Interface (hereinafter called GSP). He can then define a focus position FP in the overview OV by marking, for example by a mouse click, a desired position in the overview OV.

In a step S4 an operating mode OM is determined which is adjusted for the selected scanning protocol SP. For example a global positioning parameter of the scanning program SP can be read out for this purpose. Within the context of the invention it will hereinafter be assumed for the selected scanning protocol SP that an operating mode OM is adjusted which allows scans in which the examination couch 14, 16 does not have to be adjusted to the isocenter couch position ICP, for example if the scanning program relates to a body region of the patient 8 which even with a couch position CP that differs from the isocenter couch position ICP in the context of a previously defined tolerance delivers sufficiently good image quality. An operating mode OM of this kind is for example the LOC mode. In contrast to this, the ISO mode is an operating mode in which the isocenter couch position ICP has to be adjusted in each scanning step.

According to an embodiment of the invention, an optional step S5 can be provided in which the user can adapt or change the operating mode OM of the selected scanning protocol SP. In this ISO/LOC interaction step the user therefore has the option of switching between various operating modes via the adaptation of the global positioning parameter of the scanning program during the course of protocol planning. In particular, he can switch from a preadjusted ISO mode to an LOC mode to achieve an embodiment of an inventive display of the overview in the GSP according to step S6. This kind of change in the positioning parameter alters the operating mode OM for all subsequent scanning steps. Step S5 can take place for example by way of a drop-down menu of the GSP.

In a step S6, according to an embodiment of the invention, an overview OV is displayed, showing the body region to be examined in the GSP comprising the selection range SR to be imaged with the planned scanning protocol SP, and the focus position FP is displayed. FIG. 3 illustrates a corresponding display. The body region to be examined is here the head or neck of the patient 8. The focus position FP is illustrated in the display as a line running transversely to the longitudinal axis of the patient 8, in other words, transversely to the adjustment direction (Z-axis) of the examination couch 14, 16. Furthermore, the focus position FP is marked by means of an indicator T, arranged at the edge of the overview, in the form of a triangle whose one corner points to the Z-position of the focus position line.

According to an embodiment of the invention, in the display the selection range SR is aligned from the outset with the focus position FP. In other words, the selection range SR is arranged in such a way that for example a slice group center incorporated by the selection range SR and which runs through a center position CENP of the selection range, is located above the focus position FP. In addition, in the present case the display of the overview OV also comprises a display of the isocenter position IP, illustrated by a dot-dash line.

In other words, a position parameter is inventively provided on the routine parameter map of the selected scanning protocol SP, which indicates aligned with which position a selection range SR or a slice group center should be displayed during protocol planning. This position parameter is inventively set by default to ‘focus position’. In the individual case it can be provided that the user adapts the position parameter by way of a drop-down menu and selects for example the isocenter position IP or any other desired position as the display position.

In a subsequent step S7 the user can select a changed focus position CFP via the GSP directly in the overview OV. For this purpose, he can for example shift the selection range SR along the Z-axis until its center CENP comes to rest at a desired position, the changed focus position CFP. A shift can additionally or alternatively also occur in a different spatial direction X or Y. The changed focus position CFP can result moreover due to a rotation or tilting of the selection range SR about a center point CENP.

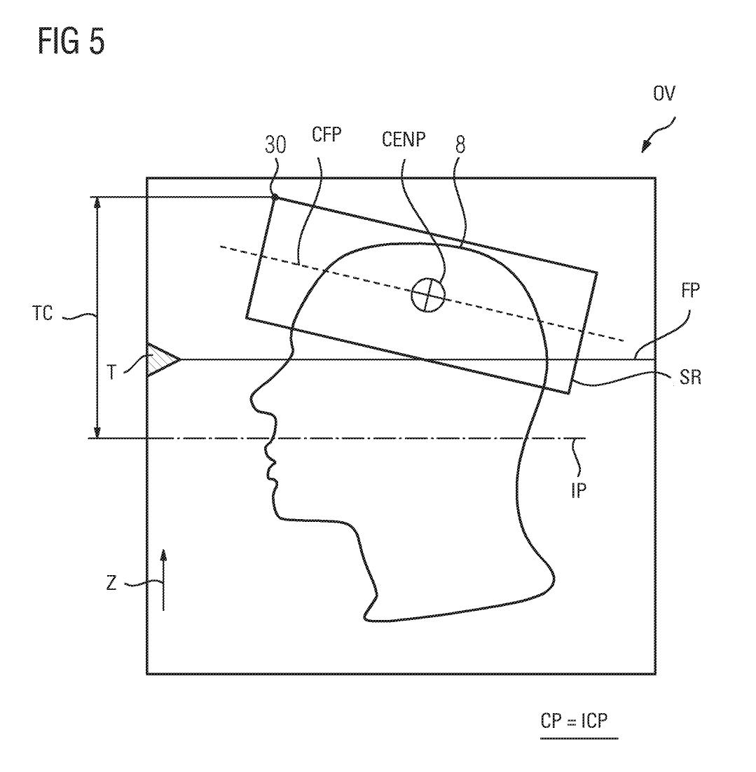

Alternatively, the user can also input coordinates or a degree for a rotation, after which the selection range SR is then shifted or rotated, for example about the center CENP. Generally speaking, the changed focus position CFP is very close, in any case much closer than the isocenter position IP, to the focus position FP. In other words, the required manipulations by the user are only slight. In a further alternative for step S7, as frequently occurs in practice, the user can the confirm the existing focus position FP. In other words, in this alternative the changed focus position CFP matches the focus position FP. This is likewise shown in FIG. 3. A shift of the selection range SR is superfluous, and this simplifies and expedites protocol planning. By contrast, FIG. 4 and FIG. 5 show a display of an overview OV in which the selection range SR or the changed focus position CFP has been shifted with respect to the focus position FP according to a user input.

A test criterion TC is checked in a step S8. The test criterion TC comprises or takes into account, as illustrated in FIG. 3 and FIG. 4 by a double-ended arrow, a distance measurement between isocenter position IP and changed focus position CFP. For this purpose the distance between isocenter position IP and changed focus position CFP is determined and compared with an upper limit for the distance and a result derived.

The test criterion TC can alternatively or additionally, as is also illustrated in FIG. 5 by a double-ended arrow, take into account a distance measurement between isocenter position IP and a position 30 that is as far away as possible therefrom within the selection range SR. For this purpose the distance between isocenter position IP and the position 30 as far away as possible is determined and compared with an upper limit for this distance and a result derived. The upper limit can in each case firstly depend on the selected scanning protocol, the medical issue, the body region considered, or the like. Secondly, the upper limit can also be derived from technical features of the tomograph 2 itself, for example the extent of the essentially homogeneous magnetic field range within the tunnel 6. In this respect the upper limit can also comprise information about the imaging quality of the tomograph 2. A respective value for the upper limit can be retrievably stored for example in the storage device 18 of the arithmetic unit 12 or on a central storage device, for example of a hospital.

In a step S9 the user is shown the test result in respect of the changed focus position CFP via the GSP. If the test shows that the distance between isocenter position IP and changed focus position CFP or furthest-away position 30 of the selection range SR is smaller than or equal to the upper limit, the focus couch position FCP follows as the couch position CP to be adjusted for the scan. In other words, the distance of isocenter position IP and changed focus position CFP lies within the tolerance with acceptable image quality. In this case, as applicable in FIG. 3 and FIG. 4, the triangle T marking the focus position FP in the overview OV is for example shown filled with color (hatching).

If, by contrast, the result of the test is greater than the upper limit, the isocenter couch position ICP follows as the couch position CP to be adjusted for the scan. In other words, the distance lies outside of the tolerance; an image acquisition would supply an unacceptable image quality here. In this case the triangle T marking the focus position FP in the overview OV is for example shown empty, in other words shown only as a boundary line, as illustrated in FIG. 5. The user can therefore directly discern which couch position CP would be adjusted for the previously selected, changed focus position CFP. If the test result illustrated in this way does not match the wishes of the user, then by way of a repetition loop (broken arrow in FIG. 2) he has the option of repeating steps S7, s8 and S9, and, more precisely, until he obtains the desired test result.

In a step S10 a control signal corresponding to the test result is generated for the tomograph 2 and transmitted thereto for adjustment of the couch position CP either to the isocenter couch position ICP or the focus couch position FCP, and the couch position CP is adjusted accordingly.

The scan is hereinafter carried out with this couch position CP for the scanning step being considered. The described method can thereafter be repeated for each further scanning step.

To summarize, one possible workflow could therefore be as follows: definition of an isocenter position IP via lasers, for example on the heart of a patient 8. Since the laser marking only allows limited accuracy, however, a focus position FP is defined or rendered more precise in a GSP by means of focus-triangle T following the first scan of a localizer image or an overview OV. A position parameter on the parameter map is inventively adjusted to the focus position FP. All protocols SP to be scanned thereafter are then opened in the GSP directly at the focus position FP during planning and no longer as previously at the isocenter position IP. This is advantageous insofar as workflows, which use various couch positions CP, can be applied in such a way that slice groups no longer have to be shifted individually in the direction of the focus position FP for each scanning step.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for adjusting a couch position for a couch of a tomograph for scanning a body region of an examination object situated on the couch, the method comprising:
acquiring a user selection of a scanning protocol from a plurality of possible scanning protocols;
acquiring a user input in respect of an isocenter position;
acquiring a user input in respect of a focus position;
displaying an overview of the body region to be examined, including a selection range and an indicator of the focus position relative to the body region, the selection range being displayable aligned with the focus position;
acquiring a user input in respect of a changed focus position;
aligning the selection range with the changed focus position; and
adjusting the couch position to a focus couch position corresponding to the focus position upon the changed focus position meeting a test criterion, and adjusting to an isocenter couch position corresponding to the isocenter position upon the changed focus position not meeting the test criterion.

2. The method of claim 1, wherein the test criterion takes into account a distance measurement between the changed focus position and the isocenter position and is met upon the distance measurement not exceeding a previously defined upper limit.

3. The method of claim 1, wherein the test criterion takes into account a distance measurement between a point of the selection range relatively furthest away from the isocenter position and the isocenter position and wherein the test criterion is met upon the distance measurement not exceeding a previously defined upper limit.

4. The method of claim 2, wherein the previously defined upper limit is dependent on at least one of an item of anatomical information and an item of information in respect of a homogeneity of a magnetic field of the tomograph.

5. The method of claim 1, wherein the overview of the body region to be examined is displayed with a selection range aligned with the focus position upon an operating mode, adjusted for the scanning protocol selected, providing for the overview to be displayed with the selection range aligned with the focus position upon an operating mode.

6. The method of claim 5, wherein the overview of the body region to be examined is displayed with a selection range aligned with a position differing from the focus position corresponding to a user input, upon the adjusted operating mode allows for the overview to be displayed with the position differing from the focus position corresponding to a user input.

7. The method of claim 6, wherein the position differing from the focus position is the isocenter position or any previously defined position within the body region to be examined.

8. The method of claim 1, further comprising
acquiring a user input in respect of an adaptation of an operating mode adjusted for the selected scanning protocol; and
adapting the operating mode corresponding to the user input acquired.

9. The method of claim 1, wherein the displaying of the overview of the body region to be examined includes displaying an indicator conveying the couch position to be adjusted as a function of the test criterion.

10. An arithmetic unit for adjusting a couch position for a couch of a tomograph for examining a body region of an examination object situated on the couch, the arithmetic unit comprising:
a memory storing program computer-readable instructions; and
one or more processors configured to execute the instructions such that the one or more processors are configured to,
acquiring a user selection of a scanning protocol from a plurality of possible scanning protocols,
acquiring a user input in respect of an isocenter position,
acquiring a user input in respect of a focus position,
displaying an overview of the body region to be examined, including a selection range and an indicator of the focus position relative to the body region, the selection range being displayable aligned with the focus position,
acquiring a user input in respect of a changed focus position,
aligning the selection range with the changed focus position, and
adjusting the couch position to a focus couch position corresponding to the focus position upon the changed focus position meeting a test criterion, and adjusting to an isocenter couch position corresponding to the isocenter position upon the changed focus position not meeting the test criterion.

11. A non-transitory computer program data carrier storing program code to carry out the method of claim 1 when the program code is run on a computer.

12. A non-transitory computer-readable data medium storing program code of a computer program to carry out the method of claim 1 when the program code is run on a computer.

13. A tomograph, usable in adjusting a couch position for a couch for examining a body region of an examination object situated on the couch, the tomograph comprising:
the arithmetic unit of claim 10.

14. The method of claim 3, wherein the previously defined upper limit is dependent on at least one of an item of anatomical information and an item of information in respect of a homogeneity of a magnetic field of the tomograph.

15. The method of claim 2, wherein the overview of the body region to be examined is displayed with a selection range aligned with the focus position upon an operating mode, adjusted for the scanning protocol selected, providing for the overview to be displayed with the selection range aligned with the focus position upon an operating mode.

16. The method of claim 15, wherein the overview of the body region to be examined is displayed with a selection range aligned with a position differing from the focus position corresponding to a user input, upon the adjusted operating mode allows for the overview to be displayed with the position differing from the focus position corresponding to a user input.

17. The method of claim 3, wherein the overview of the body region to be examined is displayed with a selection range aligned with the focus position upon an operating mode, adjusted for the scanning protocol selected, providing for the overview to be displayed with the selection range aligned with the focus position upon an operating mode.

18. The method of claim 17, wherein the overview of the body region to be examined is displayed with a selection range aligned with a position differing from the focus position corresponding to a user input, upon the adjusted operating mode allows for the overview to be displayed with the position differing from the focus position corresponding to a user input.

19. The method of claim 2, further comprising
acquiring a user input in respect of an adaptation of an operating mode adjusted for the selected scanning protocol; and
adapting the operating mode corresponding to the user input acquired.

20. The method of claim 2, wherein the displaying of the overview of the body region to be examined includes displaying an indicator conveying the couch position to be adjusted as a function of the test criterion.

21. The method of claim 3, further comprising
acquiring a user input in respect of an adaptation of an operating mode adjusted for the selected scanning protocol; and
adapting the operating mode corresponding to the user input acquired.

22. The method of claim 3, wherein the displaying of the overview of the body region to be examined includes displaying an indicator conveying the couch position to be adjusted as a function of the test criterion.
